(19) United States
(12) Patent Application Publication
(10) Pub. No.: US 2010/0048611 A1
(43) Pub. Date: Feb. 25, 2010

(54) TETRAHYDROPYRROLOPYRIDINE, TETRAHYDROPYRAZOLOPYRIDINE, TETRAHYDRO-IMIDAZOPYRIDINE AND TETRAHYDROTRIAZOLOPYRIDINE DERIVATIVES AND USE THEREOF

(75) Inventors: Susanne Röhrig, Hilden (DE); Mario Jeske, Solingen (DE); Elisabeth Perzborn, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer Healthcare AG, Leverkusen (DE)

(21) Appl. No.: 12/301,757

(22) PCT Filed: May 26, 2007
(86) PCT No.: PCT/EP2007/004707

§ 371 Date: Nov. 10, 2009

(30) Foreign Application Priority Data
May 31, 2006 (DE) 102006025315.9

(51) Int. Cl. A61K 31/437; C07D 471/04; A61P 7/02

(52) U.S. Cl. 514/303; 546/120

(57) ABSTRACT

The invention relates to novel tetrahydropyrrolopyridine, tetrahydropyrazolopyridine, tetrahydroimidazopyridine and tetrahydrotriazolopyridine derivatives, to processes for their preparation, to their use for the treatment and/or prophylaxis of diseases and to their use for preparing medicaments for the treatment and/or prophylaxis of diseases, in particular of thromboembolic disorders

The invention relates to novel tetrahydropyrrolopyridine, tetrahydropyrazolopyridine, tetrahydroimidazopyridine and tetrahydrotriazolopyridine derivatives, to processes for their preparation, to their use for the treatment and/or prophylaxis of diseases and to their use for preparing medicaments for the treatment and/or prophylaxis of diseases, in particular of thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here factor Xa, which is formed from the proenzyme factor X, plays a key role, since it connects the two coagulation paths. The activated serine protease Xa cleaves prothrombin to thrombin. The resulting thrombin, in turn, cleaves fibrinogen to fibrin. Subsequent crosslinking of the fibrin monomers causes formation of blood clots and thus haemostasis. In addition, thrombin is a potent effector of platelet aggregation which likewise contributes significantly to haemostasis.

Haemostasis is subject to a complex regulatory mechanism. Uncontrolled activation of the coagulant system or defective inhibition of the activation processes may cause formation of local thrombi or embolisms in vessels (arteries, veins, lymph vessels) or in heart cavities. This may lead to serious thromboembolic disorders. In addition, in the case of consumption coagulopathy, hypercoagulability may—systemically—result in disseminated intravascular coagulation. Thromboembolic complications furthermore occur in microangiopathic haemolytic anaemias, extracorporeal blood circulation, such as haemodialysis, and also in connection with prosthetic heart valves.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants, i.e. substances for inhibiting or preventing blood coagulation, which are known from the prior art, have various, often grave disadvantages. Accordingly, in practice, an efficient treatment method or prophylaxis of thromboembolic disorders is very difficult and unsatisfactory.

In the therapy and prophylaxis of thromboembolic disorders, use is firstly made of heparin, which is administered parenterally or subcutaneously. Owing to more favourable pharmacokinetic properties, preference is nowadays more and more given to low-molecular-weight heparin; however, even with low-molecular-weight heparin, it is not possible to avoid the known disadvantages described below, which are involved in heparin therapy. Thus, heparin is ineffective when administered orally and has a relatively short half-life. Since heparin inhibits a plurality of factors of the blood coagulation cascade at the same time, the action is non-selective. Moreover, there is a high risk of bleeding; in particular, brain haemorrhages and gastrointestinal bleeding may occur, which may result in thrombopenia, drug-induced alopecia or osteoporosis [Pschyrembel, Klinisches Wörterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”].

A second class of anticoagulants are the vitamin K antagonists. These include, for example, 1,3-indanediones, and especially compounds such as warfarin, phenprocoumon, dicumarol and other coumarin derivatives which inhibit the synthesis of various products of certain vitamin K-dependent coagulation factors in the liver in a non-selective manner. Owing to the mechanism of action, however, the onset of the action is very slow (latency to the onset of action 36 to 48 hours). It is possible to administer the compounds orally; however, owing to the high risk of bleeding and the narrow therapeutic index, a time-consuming individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a novel therapeutic approach for the treatment and prophylaxis of thromboembolic disorders has been described. This novel therapeutic approach aims to inhibit factor Xa. Because of the central role which factor Xa plays in the blood coagulation cascade, factor Xa is one of the most important targets for anticoagulants [J. Hauptmann, J. Sturzebecher, Thrombosis Research 1999, 93, 203; S. A. V. Raghavan, M. Dikshit, “Recent advances in the status and targets of antithrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for antithrombotic therapy” Drugs Fut. 2003, 28, 355-370; L.-A. Linkins, J. I. Weitz, “New anticoagulant therapy” Annu. Rev. Med. 2005, 56, 63-77; A. Casimiro-Garcia et al., “Progress in the discovery of Factor Xa inhibitors” Expert Opin. Ther. Patents 2006, 15, 119-145].

It has been shown that, in animal models, various both peptidic and nonpeptidic compounds are effective as factor Xa inhibitors. A large number of direct factor Xa inhibitors is already known [J. M. Walenga, W. P. Jeske, D. Hoppensteadt, J. Fareed, “Factor Xa Inhibitors: Today and beyond” Curr. Opin. Investig. Drugs 2003, 4, 272-281; J. Ruef, H. A. Katus, “New antithrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469]. Nonpeptidic low-molecular-weight factor Xa inhibitors are also described, for example, in WO 03/026652 and WO 03/049681.

It is an object of the present invention to provide novel alternative compounds having a comparable or improved activity and better solubility in aqueous solutions, for controlling disorders, in particular thromboembolic disorders, in humans and animals.

The invention provides compounds of the formula

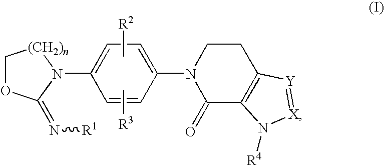

in which
n represents the number 1, 2 or 3,
X represents N or CH,
Y represents N or CR⁵,
   where
   R⁵ represents hydrogen, cyano, trifluoromethyl, trifluoromethoxy, hydroxy, hydroxymethyl, hydroxyethyl, amino, aminomethyl, aminoethyl, C₁-C₄-alkyl, C₁-C₄-alkoxy, C₁-C₄-alkoxymethyl, C₁-C₄-alkoxyethyl, C₁-C₄-alkylamino, C₁-C₄-alkylaminomethyl, C₁-C₄-alkylaminoethyl, C₃-C₆-cycloalkyl, hydroxycarbonyl, hydroxycarbonylmethyl, hydroxycarbonylethyl, aminocarbonyl, aminocarbonylmethyl, aminocarbonylethyl, C₁-C₄-alkoxycarbonyl, C₁-C₄-alkoxycarbonylmethyl, C₁-C₄-alkoxycarbonylethyl, C₁-C₄-alkylcarbonyloxy, C₁-C₄-alkylaminocarbonyl, C₁-C₄-alkylaminocarbonylmethyl, C₁-C₄-alkylaminocarbonylethyl, C₁-C₄-alkylcarbonylamino, C₁-C₄-alkylcarbonylaminomethyl, C₁-C₄-alkylcarbonylaminoethyl, C₁-C₄-alkylcarbonyl(C₁-C₄-alkyl)amino, C₁-C₄-alkylcarbonyl(C₁-C₄-alkyl)aminomethyl, C₁-C₄-alkylcarbonyl(C₁-C₄-alkyl)aminoethyl, aminosulphonyl, C₁-C₄-alkylaminosulphonyl, C₁-C₄-alkylsulphonyl or tetrazolyl,
R¹ represents hydrogen, cyano, hydroxy, C₁-C₄-alkyl, C₁-C₄-alkylcarbonyl, C₃-C₆-cycloalkylcarbonyl, phenylcarbonyl, 4- to 7-membered heterocycle carbonyl or 5- or 6-membered heteroarylcarbonyl,
R² represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R³ represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R⁴ represents phenyl, pyridyl, pyrazinyl, pyrimidinyl or pyridazinyl,
   where phenyl is substituted by a substituent R⁶ and a substituent R⁷,
      where
      R⁶ represents hydrogen, fluorine, chlorine, cyano, amino, ethynyl, aminomethyl, C₁-C₄-alkyl, C₁-C₄-alkoxy, C₃-C₆-cycloalkyl, C₃-C₆-cycloalkyloxy, aminocarbonyl, C₁-C₄-alkylaminocarbonyl, aminosulphonyl or methylcarbonylaminosulphonyl,
      and
      R⁷ represents hydrogen, fluorine, chlorine, cyano, trifluoromethyl, trifluoromethoxy, C₁-C₄-alkyl, C₁-C₄-alkoxy or C₃-C₆-cycloalkyl, or C₃-C₆-cycloalkyloxy,
   and
   where pyridyl, pyrazinyl, pyrimidinyl and pyridazinyl are substituted by a substituent R⁸ and/or a substituent R⁹ or by two different substituents R⁸ or by two different substituents R⁹,
      where
      R⁸ is attached to a carbon atom which is not adjacent to a nitrogen atom in the ring and represents hydrogen, fluorine, chlorine, cyano, amino, ethynyl, aminomethyl, C₁-C₄-alkyl, C₁-C₄-alkoxy, C₃-C₆-cycloalkyl or C₃-C₆-cycloalkyloxy,
      and
      R⁹ is attached to a carbon atom which is adjacent to a nitrogen atom in the ring and represents hydrogen, amino, ethynyl, C₁-C₄-alkyl, C₁-C₄-alkylamino, C₃-C₆-cycloalkyl or C₃-C₆-cycloalkylamino,
         and their salts, their solvates and the solvates of their salts.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by formula (I), mentioned below as embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

Alkyl per se and “alk” and “alkyl” in alkoxy alkylamino, alkylcarbonyl alkoxycarbonyl alkylcarbonyloxy alkylaminocarbonyl alkylcarbonylamino alkylaminosulphonyl and alkylsulphonyl represents a straight-chain or branched alkyl radical having generally 1 to 4, preferably 1 or 2, carbon atoms, by way of example and by way of preference methyl, ethyl, n-propyl, isopropyl and tert-butyl.

By way of example and by way of preference, alkoxy represents methoxy, ethoxy, n-propoxy, isopropoxy and tert-butoxy.

Alkylamino represents an alkylamino radical having one or two alkyl substituents (selected independently of one another), by way of example and by preference methylamino, ethylamino, n-propylamino, isopropylamino, tert-butylamino, N,N-dimethylamino, N,N-diethylamino, N-ethyl-N-methylamino, N-methyl-N-n-propylamino, N-isopropyl-N-n-propylamino and N-tert-butyl-N-methylamino.

By way of example, C₁-C₃-alkylamino represents a monoalkylamino radical having 1 to 3 carbon atoms or represents a dialkylamino radical having in each case 1 to 3 carbon atoms per alkyl substituent.

By way of example and by way of preference, alkylcarbonyl represents methylcarbonyl, ethylcarbonyl, n-propylcarbonyl, isopropylcarbonyl and tert-butylcarbonyl.

By way of example and by way of preference, alkoxycarbonyl represents methoxycarbonyl, ethoxycarbonyl, n-propoxycarbonyl, isopropoxycarbonyl and tert-butoxycarbonyl.

By way of example and by way of preference, alkylcarbonyloxy represents methylcarbonyloxy, ethylcarbonyloxy, n-propylcarbonyloxy, isopropylcarbonyloxy and tert-butylcarbonyloxy.

Alkylaminocarbonyl represents an alkylaminocarbonyl radical having one or two alkyl substituents (selected independently of one another), by way of example and by way of preference methylaminocarbonyl, ethylaminocarbonyl, n-propylaminocarbonyl, isopropylaminocarbonyl, tertbutylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N-tert-butyl-N-methylaminocarbonyl. By way of example, C₁-C₃-alkylaminocarbonyl represents a monoalkylaminocarbonyl radical having 1 to 3 carbon atoms or represents a dialkylaminocarbonyl radical having in each case 1 to 3 carbon atoms per alkyl substituent.

By way of example and by way of preference, alkylcarbonylamino represents methylcarbonylamino, ethylcarbonylamino, n-propylcarbonylamino, isopropylcarbonylamino and tert-butylcarbonylamino.

Alkylaminosulphonyl represents an alkylaminosulphonyl radical having one or two alkyl substituents (chosen independently of one another), by way of example and by way of preference methylaminosulphonyl, ethylaminosulphonyl, n-propylaminosulphonyl, isopropylaminosulphonyl, tert-butylaminosulphonyl, N,N-dimethylaminosulphonyl, N,N-diethylaminosulphonyl, N-ethyl-N-methylaminosulphonyl, N-methyl-N-n-propylaminosulphonyl, N-isopropyl-N-n-propylaminosulphonyl and N-tert-butyl-N-methylaminosulphonyl. C₁-C₃-Alkylaminosulphonyl represents, for example, a monoalkylaminosulphonyl radical having 1 to 3 carbon atoms or represents a dialkylaminosulphonyl radical having in each case 1 to 3 carbon atoms per alkyl substituent.

By way of example and by way of preference, alkylsulphonyl represents methylsulphonyl, ethylsulphonyl, n-propylsulphonyl, isopropylsulphonyl and tert-butylsulphonyl.

Cycloalkyl represents a cycloalkyl group having generally 3 to 6 carbon atoms, preferably 3 to 5 carbon atoms, by way of example and by way of preference cyclopropyl, cyclobutyl, cyclopentyl and cyclohexyl.

Heterocyclyl represents a monocyclic heterocyclic radical having generally 4 to 7 ring atoms and up to 3, preferably up to 2, heteroatoms and/or heterogroups from the group consisting of N, O, S, SO, SO₂. The heterocyclyl radicals can be saturated or partially unsaturated. Preference is given to 5- to 7-membered monocyclic saturated heterocyclyl radicals having up to two heteroatoms from the group consisting of O, N and S, such as, by way of example and by way of preference, tetrahydrofuranyl, pyrrolidinyl, pyrrolinyl, piperidinyl, tetrahydropyranyl, piperazinyl, morpholinyl and perhydroazepinyl.

Heteroaryl represents an aromatic monocyclic radical having 5 or 6 ring atoms and up to 4 heteroatoms from the group consisting of S, O and N, by way of example and by way of preference thienyl, furyl, pyrrolyl, thiazolyl, oxazolyl, isoxazolyl, isothiazolyl, imidazolyl, pyrazolyl, pyridyl, pyrimidinyl, pyridazinyl and pyrazinyl.

If radicals in the compounds according to the invention are substituted, the radicals can, unless specified otherwise, be mono- or polysubstituted. In the context of the present invention, the meanings of all radicals which occur more than once are independent of one another. Substitution with one, two or three identical or different substituents is preferred. Very particular preference is given to substitution with one substituent.

Preference is given to compounds of the formula (I) in which
n represents the number 1, 2 or 3,
X represents N or CH,
Y represents N or CR⁵,
   where
   R⁵ represents hydrogen, cyano, trifluoromethyl, hydroxymethyl, aminomethyl, C₁-C₄-alkyl, C₁-C₄-alkoxy, C₁-C₄-alkoxymethyl, C₁-C₄-alkylamino, C₁-C₄-alkylaminomethyl, C₃-C₆-cycloalkyl, hydroxycarbonyl, hydroxycarbonylmethyl, aminocarbonyl, aminocarbonylmethyl, C₁-C₄-alkoxycarbonyl, C₁-C₄-alkoxycarbonylmethyl, C₁-C₄-alkylaminocarbonyl, C₁-C₄-alkylaminocarbonylmethyl, C₁-C₄-alkylcarbonylamino, C₁-C₄-alkylcarbonylaminomethyl, C₁-C₄-alkylcarbonyl-(C₁-C₄-alkyl)amino, C₁-C₄-alkylcarbonyl-(C₁-C₄-alkyl)aminomethyl, aminosulphonyl, C₁-C₄-alkylaminosulphonyl, C₁-C₄-alkylsulphonyl or tetrazolyl,
R¹ represents hydrogen, cyano, hydroxy or C₁-C₄-alkyl,
R² represents hydrogen, fluorine, chlorine, cyano, hydroxy, C₁-C₄-alkyl or C₁-C₄-alkoxy,
R³ represents hydrogen, fluorine, chlorine, cyano, hydroxy, C₁-C₄-alkyl, C₁-C₄-alkoxy, C₁-C₄-alkoxymethyl, cyclopropyl, aminocarbonyl, C₁-C₄-alkoxycarbonyl or C₁-C₄-alkylaminocarbonyl,
R⁴ represents phenyl, pyridyl, pyrazinyl, pyrimidinyl or pyridazinyl,
   where phenyl is substituted by a substituent R⁶ and a substituent R⁷,
      where
      R⁶ represents hydrogen, fluorine, chlorine, cyano, ethynyl, aminomethyl, methyl, ethyl, methoxy, ethoxy, aminocarbonyl, aminosulphonyl or methylcarbonylaminosulphonyl,
      R⁷ represents hydrogen, fluorine, chlorine, cyano, trifluoromethyl, trifluoromethoxy, methyl, ethyl, methoxy or ethoxy,
   and
   where pyridyl, pyrazinyl, pyrimidinyl and pyridazinyl are substituted by a substituent R⁸ and/or a substituent R⁹ or by two different substituents R⁸ or by two different substituents R⁹,
      where
      R⁸ is attached to a carbon atom which is not adjacent to a nitrogen atom in the ring and represents hydrogen, fluorine, chlorine, cyano, ethynyl, aminomethyl, methyl, ethyl, methoxy or ethoxy,
      R⁹ is attached to a carbon atom which is adjacent to a nitrogen atom in the ring, and represents hydrogen, amino, ethynyl, methyl, ethyl, methylamino or dimethylamino,
      and
      where R⁸, if present, is attached to the para-position and R⁹, if present, is attached to the meta-position to the point of attachment of the phenyl ring,
      or
      where R⁸, if present, is attached to the meta-position and R⁹, if present, is attached to the para-position to the point of attachment of the phenyl ring,
         and their salts, their solvates and the solvates of their salts.

Preference is also given to compounds of the formula (I) in which
n represents the number 1 or 2,
X represents N,
Y represents CR⁵,
   where
   R⁵ represents hydrogen, cyano, trifluoromethyl, hydroxymethyl, aminomethyl, methoxy, methoxymethyl, methylaminomethyl, dimethylaminomethyl, hydroxycarbonyl, aminocarbonyl, methoxycarbonyl, ethoxycarbonyl, methylaminocarbonyl, dimethylaminocarbonyl or tetrazolyl,
R² represents hydrogen,
R² represents hydrogen,
R³ represents hydrogen, fluorine, chlorine, cyano, methyl, ethyl, n-propyl, methoxy, ethoxy or methoxymethyl,
R⁴ represents 4-methoxyphenyl, 3-chlorophenyl, 3-chloro-4-fluorophenyl, 4-chlorophenyl, 3-methylphenyl, 3-methyl-4-fluorophenyl, 4-methylphenyl, 4-ethylphenyl, 3-aminomethylphenyl, 3-aminomethyl-4-fluorophenyl, 3-aminocarbonylphenyl, 5-chloropyridin-2-yl, 5-methylpyridin-2-yl, 5-ethylpyridin-2-yl, 5-methoxypyridin-2-yl, 5-chloropyrimidin-2-yl, 5-methylpyrimidin-2-yl, 5-ethylpyrimidin-2-yl, 5-methoxypyrimidin-2-yl, 6-methylpyridin-3-yl, 6-ethylpyridin-3-yl, 6-methylpyridazin-3-yl or 6-ethylpyridazin-3-yl,
   and their salts, their solvates and the solvates of their salts.

Preference is also given to compounds of the formula (I) in which
n represents the number 1,
X represents N,
Y represents CR⁵,
   where
   R⁵ represents aminocarbonyl, methoxycarbonyl or ethoxycarbonyl,
R¹ represents hydrogen,
R² represents hydrogen,
R³ represents hydrogen, fluorine, chlorine, cyano, methyl or methoxy,
R⁴ represents 4-methoxyphenyl, and their salts, their solvates and the solvates of their salts.

Preference is also given to compounds of the formula (I) in which n represents the number 1.

Preference is also given to compounds of the formula (I) in which X represents N and Y represents CR⁵.

Preference is also given to compounds of the formula (I) in which X represents N, Y represents CR⁵ and R⁵ represents aminocarbonyl, methoxycarbonyl or ethoxycarbonyl.

Preference is also given to compounds of the formula (I) in which R¹ represents hydrogen.

Preference is also given to compounds of the formula (I) in which R² represents hydrogen.

Preference is also given to compounds of the formula (I) in which R³ represents hydrogen, fluorine, chlorine, cyano, methyl or methoxy.

Preference is also given to compounds of the formula (I) in which R³ represents hydrogen.

Preference is also given to compounds of the formula (I) in which R² and R³ represent hydrogen.

Preference is also given to compounds of the formula (I) in which R⁴ represents 4-methoxyphenyl, 3-chlorophenyl, 4-chlorophenyl, 3-methylphenyl, 4-methylphenyl or 4-ethylphenyl.

Preference is also given to compounds of the formula (I) in which R⁴ represents 4-methoxyphenyl.

The individual radical definitions given in the respective combinations or preferred combinations of radicals are, independently of the particular given combinations of radicals, also replaced by any radical definitions of other combinations.

Very particular preference is given to combinations of two or more of the preferred ranges mentioned above.

The invention furthermore provides a process for preparing the compounds of the formula (I), or their salts, their solvates or the solvates of their salts, wherein

[A] the compounds of the formula

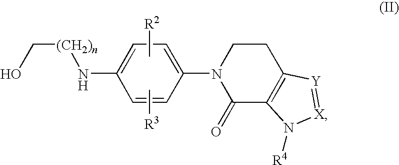

in which n, X, Y, R², R³ and R⁴ have the meaning given above,
are reacted in an inert solvent in the presence of an acid with cyanogen bromide with compounds of the formula (I) in which R¹ represents hydrogen,
or
[B] the compounds of the formula

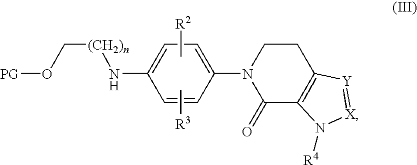

in which n, X, Y, R², R³ and R⁴ have the meaning given above, and
PG represents a hydroxyl protective group, preferably trimethylsilyl or tert-butyldimethylsilyl, are, in a three-step process, initially reacted in an inert solvent with cyanogen bromide, preferably in the presence of a base, to give compounds of the formula

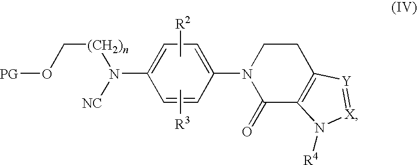

in which n, X, Y, R², R³ and R⁴ have the meaning given above and
PG represents a hydroxyl protective group, preferably trimethylsilyl or tert-butyldimethylsilyl, and then, by removal of the protective group PG converted into compounds of the formula

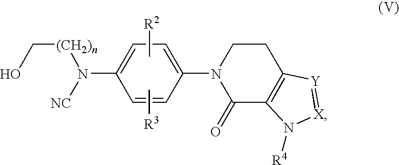

in which n, X, Y, R², R³ and R⁴ have the meaning given above,
and, in the third step the compounds of the formula (V) are cyclized in an inert solvent in the presence of an acid to give compounds of the formula (I) in which R¹ represents hydrogen,
or
[C] the compounds of the formula (II) are, in the first step, reacted with compounds of the formula

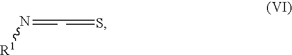

in which
R¹ represents C₁-C₄-alkyl, C₁-C₄-alkylcarbonyl, C₃-C₆-cycloalkylcarbonyl, phenylcarbonyl, 4 to 7-membered heterocyclylcarbonyl or 5- or 6-membered heteroarylcarbonyl,
   and, in the second step, cyclized,
   or
   [D] the compounds of the formula (II) are reacted with compounds of the formula

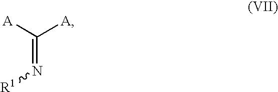

in which
R¹ represents cyano or C₁-C₄-alkyl, and
A represents a leaving group, preferably phenoxy or methylthio,
   or
   [E] the compounds of the formula (I) in which R¹ represents hydrogen are reacted with hydroxylamine hydrochloride to give compounds of the formula (I) in which R′ represents hydroxyl.

If appropriate, the compounds of the formula (I) in which R¹ represents hydrogen can be converted with the appropriate solvents and/or bases or acids into their salts, their solvates and/or the solvates of their salts.

The free base of the salts can be obtained, for example, by chromatography on a reversed-phase column using an acetonitrile/water gradient with an added base, in particular by using an RP18 Phenomenex Luna C18(2) column and diethylamine base, or by dissolving the salts in an organic solvent and extracting with aqueous solutions of basic salts such as sodium bicarbonate.

The invention furthermore provides a process for preparing the compounds of the formula (I) of their solvates wherein salts of the compounds or solvates of the salts of the compounds are converted into the compounds by chromatography with an added base.

The reaction according to process [A] is generally carried out in inert solvents, preferably in a temperature range of from −20° C. to 50° C. at atmospheric pressure.

Inert solvents are, for example, tetrahydrofuran, dichloromethane or acetonitrile or mixtures of these solvents.

Acids are, for example, strong inorganic or organic acids, such as hydrogen fluoride, hydrogen chloride, hydrogen bromide, methanesulphonic acid, trifluoromethanesulphonic acid or trifluoroacetic acid.

The reaction of the first step according to process [B] is generally carried out in an inert solvent, preferably in a temperature range of from −20° C. to 50° C. at atmospheric pressure.

Inert solvents are, for example, tetrahydrofuran, dichloromethane or acetonitrile or mixtures of these solvents.

Bases are, for example, inorganic bases, such as alkali metal or alkaline earth metal carbonates or bicarbonates, such as lithium carbonate, sodium carbonate, potassium carbonate, calcium carbonate or caesium carbonate or sodium bicarbonate or potassium bicarbonate, or alkali metal hydrides, such as sodium hydride.

The removal of trimethylsilyl or tert-butyldimethylsilyl as preferred hydroxyl protective groups (PG) in the second step according to process [B] is generally carried out in tetrahydrofuran as solvent, preferably with the aid of tetra-n-butylammonium fluoride (TBAF), preferably in a temperature range of from 0° C. to 40° C. at atmospheric pressure.

The reaction of the third step according to process [B] is generally carried out in inert solvents, preferably in a temperature range of from −20° C. to 50° C. at atmospheric pressure.

Inert solvents are, for example, tetrahydrofuran, dichloromethane or acetonitrile or mixtures of these solvents.

Acids are, for example, strong inorganic or organic acids, such as hydrogen fluoride, hydrogen chloride, hydrogen bromide, methanesulphonic acid, trifluoromethanesulphonic acid or trifluoroacetic acid.

The reaction of the second and third step according to process [B] is particularly preferably carried out using an acid-labile hydroxyl protective group, such as, for example, trimethylsilyl or tertbutyldimethylsilyl, in the presence of an excess of an acid as a one-pot reaction, in inert solvents, preferably in a temperature range of from −20° C. to 50° C. at atmospheric pressure, without isolation of the intermediate of the compounds of the formula (V).

Inert solvents are, for example, tetrahydrofuran, dichloromethane or acetonitrile or mixtures of these solvents.

Acids are, for example, strong inorganic or organic acids, such as hydrogen fluoride, hydrogen chloride, hydrogen bromide, methanesulphonic acid, trifluoromethanesulphonic acid or trifluoroacetic acid.

The reaction of the first step according to process [C] is generally carried out analogously to processes known from the literature, as described, for example, in A. Hetenyi et al., J. Org. Chem. 2003, 68, 2175-2182; D. Douglass, J. Am. Chem. Soc. 1934, 56, 719; F. B. Dains et al., J. Am. Chem. Soc. 1925, 47, 1981-1989 or F. B. Dains et al., J. Am. Chem. Soc. 1922, 44, 2637-2643.

The reaction of the second step according to process [C] is generally carried out analogously to processes known from the literature, as described, for example, in T. Shibanuma, M. Shiono, T. Mukaiyama, Chem. Lett. 1977, 575-576.

The reaction according to process [D] is generally carried out analogously to processes known from the literature, as described, for example, in N. Maezaki, A. Furusawa, S. Uchida, T. Tanaka, Tetrahedron 2001, 57, 9309-9316; G. Berecz, J. Reiter, G. Argay, A. Kalman, J. Heterocycl. Chem. 2002, 39, 319-326; R. Evers, M. Michalik, J. Prakt. Chem. 1991, 333, 699-710; R. Mohr, A. Buschauer, W. Schunack, Arch. Pharm. (Weinheim Ger.) 1988, 321, 221-227; P. J. Garratt et al., Tetrahedron 1989, 45, 829-834 or V. A. Vaillancourt et al., J. Med. Chem. 2001, 44, 1231-1248.

The reaction according to process [E] is generally carried out analogously to processes known from the literature, as described, for example, in G. Zinner, G. Nebel, Arch. Pharm. Ber. Dtsch. Ges. 1970, 303, 385-390.

The compounds of the formulae (VI) and (VII) are known or can be synthesized according to known processes from the appropriate starting materials.

The compounds of the formula (II) are known or can be prepared from the compounds of the formula (III) by removing the protective group PG under conditions known to the person skilled in the art.

The removal of trimethylsilyl or tert-butyldimethylsilyl as preferred hydroxyl protective groups (PG) is generally carried out in tetrahydrofuran as solvent, preferably with the aid of tetra-n-butylammonium fluoride (TBAF) or hydrogen fluoride, preferably in a temperature range of from 0° C. to 40° C. at atmospheric pressure.

The compounds of the formula (III) are known or can be prepared by reacting compounds of the formula

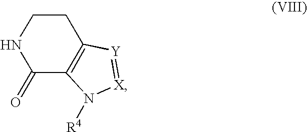

in which X, Y and R⁴ have the meaning given above,
with compounds of the formula

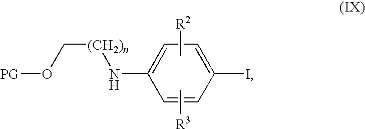

in which n, R², R³ and PG have the meaning given above,
under Ullmann reaction conditions.

The reaction is generally carried out in inert solvents with addition of a copper(I) salt, a base and a diamine ligand, preferably in a temperature range of from 60° C. to reflux of the solvent at atmospheric pressure.

Inert solvents are, for example, aproptic solvents, such as toluene, dioxane, tetrahydrofuran or dimethylformamide; preference is given to dioxane.

Copper(I) salts are, for example, copper(I) iodide, copper(I) chloride or copper(I) oxide; preference is given to copper(I) iodide.

Bases are, for example, potassium phosphate, potassium carbonate or caesium carbonate; preference is given to potassium phosphate.

Diamine ligands are, for example, 1,2-diamines, such as N,N′-dimethylethylenediamine.

The compounds of the formulae (VIII) and (IX) are known or can be synthesized by known processes from the appropriate starting materials.

The preparation of the compounds according to the invention can be illustrated by the synthesis scheme below:

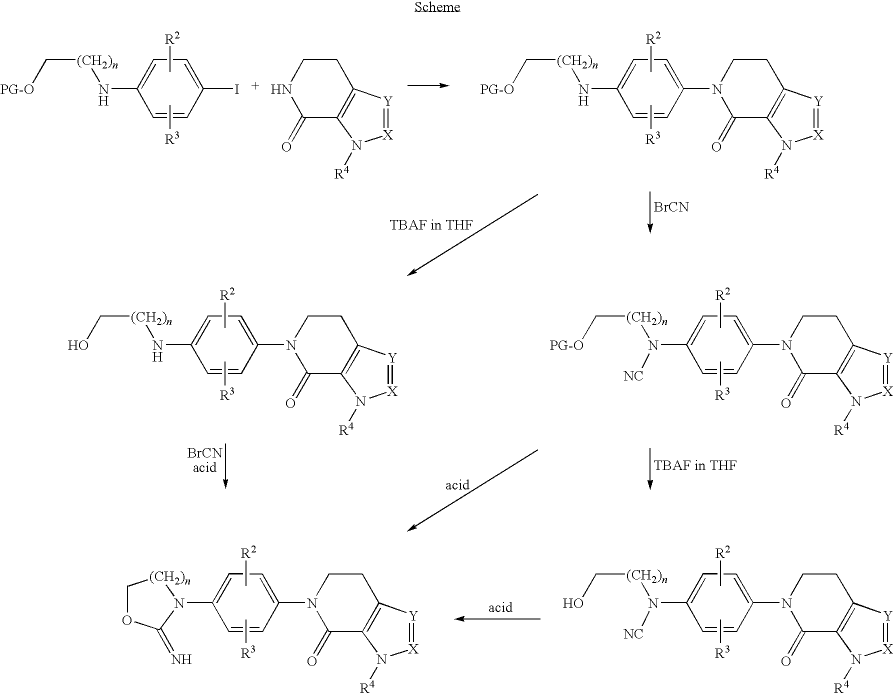

The compounds according to the invention have an unforeseeable useful pharmacological activity spectrum.

Accordingly, they are suitable for use as medicaments for the treatment and/or prophylaxis of diseases in humans and animals.

The compounds according to the invention are selective inhibitors of blood coagulation factor Xa which act in particular as anticoagulants.

In addition, the compounds according to the invention have favourable physicochemical properties, such as, for example, good solubility in water and physiological media, which is advantageous for their therapeutic application.

The present invention furthermore provides the use of the compounds according to the invention for the treatment and/or prophylaxis of disorders, preferably thromboembolic disorders and/or thromboembolic complications.

For the purposes of the present invention, “thromboembolic disorders” include in particular disorders such as ST-elevation myocardial infarction (STEMI) or non-ST-elevation myocardial infarction (non-STEMI), stable angina pectoris, unstable angina pectoris, reocclusions and restenoses after coronary interventions such as angioplasty or aortocoronary bypass, peripheral arterial occlusive diseases, pulmonary embolisms, deep vein thromboses and kidney vein thromboses, transitory ischaemic attacks and also thrombotic and thromboembolic stroke.

Accordingly, the substances are also suitable for preventing and treating cardiogenic thromboembolisms, such as, for example, brain ischaemias, stroke and systemic thromboembolisms and ischaemias, in patients having acute, intermittent or persistent cardioarrhythmias, such as, for example, atrial fibrillation, and those undergoing cardioversion, furthermore patients having heart valve disorders or having artificial heart valves. In addition, the compounds according to the invention are suitable for treating disseminated intravascular coagulation (DIC).

Thromboembolic complications furthermore occur during microangiopathic haemolytic anaemias, extracorporeal blood circulation, such as haemodialysis, and in connection with heart valve prostheses.

Moreover, the compounds according to the invention are also suitable for the prophylaxis and/or treatment of atherosclerotic vascular disorders and inflammatory disorders, such as rheumatic disorders of the locomotor apparatus, and in addition also for the prophylaxis and/or treatment of Alzheimer's disease. Moreover, the compounds according to the invention can be used for inhibiting tumour growth and formation of metastases, for microangiopathies, age-related macular degeneration, diabetic retinopathy, diabetic nephropathy and other microvascular disorders, and also for the prevention and treatment of thromboembolic complications, such as, for example, venous thromboembolisms, in tumour patients, in particular patients undergoing major surgical interventions or chemo- or radiotherapy.

The compounds according to the invention can additionally also be used for preventing coagulation ex vivo, for example for preserving blood and plasma products, for cleaning/pretreating catheters and other medical tools and instruments, for coating synthetic surfaces of medical tools and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatory effective amount of the compound according to the invention.

The present invention furthermore provides a method for preventing blood coagulation in vitro, in particular in banked blood or biological samples comprising factor Xa, which method is characterized in that an anticoagulatory effective amount of the compound according to the invention is added.

The present invention furthermore provides medicaments comprising a compound according to the invention and one or more further active compounds, in particular for the treatment and/or prophylaxis of the disorders mentioned above. The following compounds may be mentioned by way of example and by way of preference as active compounds suitable for combinations:
lipid-lowering agents, in particular HMG-CoA (3-hydroxy-3-methylglutaryl-coenzyme A) reductase inhibitors;
coronary therapeutics/vasodilators, in particular ACE (angiotensin converting enzyme) inhibitors; AII (angiotensin II) receptor antagonists; β-adrenoceptor antagonists; alpha-1-adrenoceptor antagonists; diuretics; calcium channel blockers; substances which cause an increase in the cyclic guanosine monophosphate (cGMP) concentration such as, for example, stimulators of soluble guanylate cyclase;
plasminogen activators (thrombolytics/fibrinolytics) and compounds enhancing thrombolysis/fibrinolysis, such as inhibitors of the plasminogen activator inhibitor (PAI inhibitors) or inhibitors of the thrombin-activated fibrinolysis inhibitor (TAFI inhibitors);
anticoagulants;
platelet aggregation inhibiting substances (platelet aggregation inhibitors, thrombocyte aggregation inhibitors);
fibrinogen receptor antagonists (glycoprotein-IIb/IIIa antagonists);
and also antiarrhythmics.

The present invention furthermore provides medicaments comprising at least one compound according to the invention, usually together with one or more inert non-toxic pharmaceutically acceptable auxiliaries, and their use for the purposes mentioned above.

The compounds according to the invention can act systemically and/or locally. For this purpose, they can be administered in a suitable way, such as, for example, by the oral, parenteral, pulmonary, nasal, sublingual, lingual, buccal, rectal, dermal, transdermal, conjunctival or otic route, or as implant or stent.

For these administration routes, it is possible to administer the compounds according to the invention in suitable administration forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (uncoated and coated tablets, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbarly) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for other administration routes are pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Preference is given to oral or parenteral administration, in particular oral administration.

The compounds according to the invention can be converted into the stated administration forms. This can take place in a manner known per se by mixing with inert, non-toxic, pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavour- and/or odour-masking agents.

In general, it has proved advantageous to administer on parenteral administration amounts of from about 0.001 to 1 mg/kg, preferably from about 0.01 to 0.5 mg/kg, of body weight to achieve effective results. The dosage on oral administration is from about 0.01 to 100 mg/kg, preferably about 0.01 to 20 mg/kg, and very particularly preferably 0.1 to 10 mg/kg, of body weight.

It may nevertheless be necessary, where appropriate, to deviate from the amounts mentioned, depending on the body weight, the administration route, the individual response to the active compound, the mode of preparation and the time or interval over which administration takes place. Thus, in some cases it may be sufficient to make do with less than the aforementioned minimal amount, whereas in other cases the upper limit mentioned must be exceeded. In the event of administration of larger amounts, it may be advisable to divide these into a plurality of individual doses over the day.

The invention is illustrated by the working examples below. The invention is not limited to the examples.

The percentage data in the following tests and examples are percentages by weight unless otherwise indicated; parts are parts by weight. Solvent ratios, dilution ratios and concentration data of liquid/liquid solutions are in each case based on volume.

### A. EXAMPLES

### Abbreviations

### TLC Thin-Layer Chromatography

### DCI Direct Chemical Ionization (in MS)

### DMF N,N-Dimethylformamide

DMSO Dimethyl sulphoxide
d day(s)
ee Enantiomeric excess
eq. Equivalent(s)

### ESI Electrospray Ionization (in MS)

h hour(s)

### HPLC High-Pressure, High-Performance Liquid Chromatography

### LC-MS Liquid Chromatography-coupled Mass Spectroscopy

min minute(s)

### MS Mass Spectroscopy

NMR Nuclear Magnetic Resonance spectroscopy

### RP Reversed Phase (in HPLC)

### RT Room Temperature

RT Retention time (in HPLC)
TBTU O-(benzotriazol-1-yl)-N,N,N′N′-tetramethyluronium tetrafluoroborate

### THF Tetrahydrofuran

### LC-MS and HPLC Methods

Method 1: MS instrument type: Micromass ZQ; HPLC instrument type: Waters Alliance 2795; Column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; Gradient: 0.0 min 90% A→2.5 min 30% A→3.0 min 5% A→4.5 min 5% A; flow rate: 0.0 min 1 mL/min→2.5 min/3.0 min/4.5 min 2 mL/min; oven: 50° C.; UV detection: 210 nm.

Method 2: MS instrument type: Micromass ZQ; HPLC instrument type: HP 1100 Series; UV DAD; column: Phenomenex Synergi 2μ Hydro-RP Mercury 20 mm×4 mm; mobile phase A: 1 l of water+0.5 ml of 50% strength formic acid, mobile phase B: 111 of acetonitrile+0.5 ml of 50% strength formic acid; gradient: 0.0 min 90% A→2.5 min 30% A→3.0 min 5% A→4.5 min 5% A; flow rate: 0.0 min 1 mL/min→2.5 min/3.0 min/4.5 min 2 mL/min; oven: 50° C.; UV detection: 210 nm.

Method 3: Instrument: Micromass Quattro LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 111 of acetonitrile+0.5 ml of 50% strength formic acid; gradient: 0.0 min 90% A→2.5 min 30% A→3.0 min 5% A→4.5 min 5% A; flow rate: 0.0 min 1 mL/min→2.5 min/3.0 min/4.5 min 2 ml/min; oven: 50° C.; UV detection: 208-400 nm.

Method 4: Instrument: Micromass Platform LCZ with HPLC Agilent Series 1100; column: Thermo HyPURITY Aquastar 3μ 50 mm×2.1 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 5: Instrument: HP 1100 with DAD detection; column: Kromasil 100 RP-18, 60 mm×2.1 mm, 3.5 μm; mobile phase A: 5 ml of perchloric acid (70% strength)/1 of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 mL/min; column temperature: 30° C.; UV detection: 210 nm.

### Starting Materials

### Example 1A

### Ethyl 1-(4-methoxyphenyl)-7-oxo-4,5,6,7-tetrahydro-1H-pyrazolo[3,4-c]pyridine-3-carboxylate

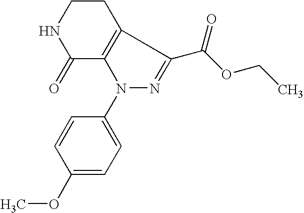

The title compound is prepared analogously to the process described in WO 03/049681 (Examples 40-44).

### Example 2A

### N-(2-{[tert-Butyl(dimethyl)silyl]oxy}ethyl)-4-iodoaniline

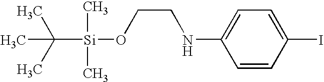

### Step a) 2-[(4-Iodophenyl)amino]ethanol

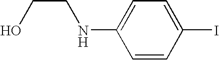

Under argon and at RT, 500 mg (8.2 mmol) of 2-aminoethanol, 5.40 g (16.4 mmol, 2 eq.) of 1,4-diiodobenzene and 1.83 ml (32.7 mmol, 4 eq.) of 1,2-ethanediol are added to a suspension of 6.95 g (32.7 mmol, 4 eq.) of potassium phosphate and 312 mg (1.6 mmol, 0.2 eq.) of copper(I) iodide in 50 ml of absolute isopropanol. The reaction mixture is stirred at 80° C. for 17 h. After cooling to RT, the reaction mixture is filtered and the residue is washed with isopropanol. The combined filtrates are concentrated under reduced pressure. The title compound is isolated by flash chromatography (silica gel, mobile phase: dichloromethane/methanol 200:1→50:1).

Yield: 2.24 g (86% pure, 89% of theory)

LC-MS (method 4): Rₜ=3.23 min;

MS (ESIpos): m/z=264 [M+H]⁺;

¹H-NMR (400 MHz, DMSO-d₆): δ=7.31 (d, 2H), 6.43 (d, 2H), 5.74 (t, 1H), 4.68 (t, 1H), 3.52 (q, 2H), 3.05 (q, 2H).

### Step b): N-(2-{[tert-Butyl(dimethyl)silyl]oxy}ethyl)-4-iodoaniline

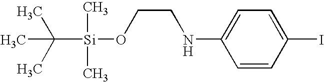

Under argon and at RT, 993 mg (14.6 mmol, 2 eq.) of imidazole and 1.32 g (8.7 mmol, 1.2 eq.) of tert-butyldimethylsilyl chloride are added to a solution of 2.23 g (86% pure, 7.3 mmol) of 2-[(4-iodophenyl)amino]ethanol in 50 ml of tetrahydrofuran. The reaction mixture is stirred at RT for 18 h, a further 549 mg (3.6 mmol, 0.5 eq.) of tert-butyldimethylsilyl chloride are added and the mixture is stirred at RT for 2 h. After addition of water/dichloromethane and phase separation, the aqueous phase is extracted twice with dichloromethane. The combined organic phases are washed with water and with saturated aqueous sodium chloride solution, dried over sodium sulphate, filtered and concentrated under reduced pressure. The title compound is isolated by flash chromatography (silica gel, mobile phase: cyclohexane/ethyl acetate 200:1→100:1).

Yield: 2.6 g (96% pure, 91% of theory)

LC-MS (method 3): Rₜ=3.45 min;

MS (ESIpos): m/z=378 [M+H]⁺;

¹H-NMR (400 MHz, DMSO-d₆): δ=7.29 (d, 2H), 6.41 (d, 2H), 5.72 (t, 1H), 3.66 (t, 2H), 3.09 (q, 2H), 0.82 (s, 9H), 0.0 (s, 6H).

### Working Examples

### Example 1

### 6-[4-(2-Imino-1,3-oxazolidin-3-yl)phenyl]-1-(4-methoxyphenyl)-7-oxo-4,5,6,7-tetrahydro-1H-pyrazolo[3,4-c]pyridine-3-carboxamide methanesulphonate

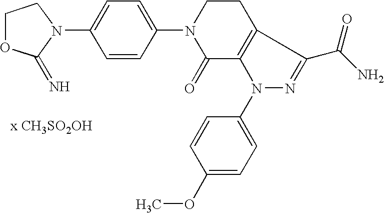

### Step a): Ethyl 6-{4-[(2-{[tert-butyl(dimethyl)silyl]oxy}ethyl)amino]phenyl}-1-(4-methoxyphenyl)-7-oxo-4,5,6,7-tetrahydro-1H-pyrazolo[3,4-c]pyridine-3-carboxylate

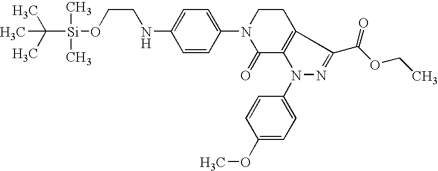

Under argon and at RT, 273 mg (96% pure, 0.70 mmol, 1 eq.) of 2-N-(2-{[tertbutyl(dimethyl)silyl]oxy}ethyl)-4-iodoaniline (Example 2A), 233 mg (0.70 mmol) of ethyl 1-(4-methoxyphenyl)-7-oxo-4,5,6,7-tetrahydro-1H-pyrazolo[3,4-c]pyridine-3-carboxylate (Example 1A) and 22 μl (0.21 mmol, 0.3 eq.) of N,N′-dimethylethylenediamine are added to a suspension of 295 mg (1.39 mmol, 2 eq.) of potassium phosphate and 26 mg (0.14 mmol, 0.2 eq.) of copper(I) iodide in 9 ml of absolute dioxane. The reaction mixture is stirred under reflux for 17 h. After cooling to RT, the reaction mixture is filtered through Celite, and the residue is washed with dioxane. The combined filtrates are concentrated under reduced pressure. The title compound is isolated by preparative RP-HPLC (CromSil C18, acetonitrile/water gradient).

Yield: 244 mg (94% pure, 58% of theory)

LC-MS (method 1): Rₜ=3.14 min;

MS (ESIpos): m/z=565 [M+H]⁺;

¹H-NMR (400 MHz, DMSO-d): δ=7.45 (d, 2H), 7.01 (d, 2H), 6.99 (d, 2H), 6.56 (d, 2H), 5.56 (t, 1H), 4.32 (q, 2H), 3.93 (t, 2H), 3.80 (s, 3H), 3.69 (t, 2H), 3.19-3.09 (m, 4H), 1.30 (t, 3H), 0.85 (s, 9H), 0.0 (s, 6H).

### Step b): 6-{4-[(2-{[tert-Butyl(dimethyl)silyl]oxy}ethyl)amino]phenyl}-1-(4-methoxyphenyl)-7-oxo-4,5,6,7-tetrahydro-1H-pyrazolo[3,4-c]pyridine-3-carboxylic acid

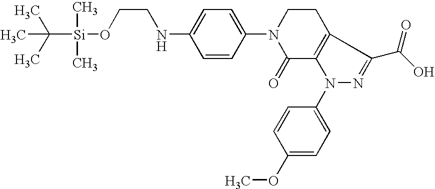

At RT, 20 mg (0.81 mmol, 2 eq.) of lithium hydroxide are added to a solution of 244 mg (94% pure, 0.41 mmol) of ethyl 6-{4-[(2-{[tert-butyl(dimethyl)silyl]oxy}ethyl)amino]phenyl}-1-(4-methoxyphenyl)-7-oxo-4,5,6,7-tetrahydro-1H-pyrazolo[3,4-c]pyridine-3-carboxylate in 12 ml of a mixture of tetrahydrofuran/water (3:1). The reaction mixture is stirred at RT for 5 h and diluted with water, and the pH is adjusted to 2 using 1 N hydrochloric acid. After addition of ethyl acetate and phase separation, the aqueous phase is extracted three times with ethyl acetate. The combined organic phases are dried over sodium sulphate, filtered and concentrated under reduced pressure. The crude product is used without further purification in the next reaction.

Yield: 220 mg (70% pure, 74% of theory)

LC-MS (method 2): Rₜ=3.06 min;

MS (ESIpos): m/z=537 [M+H]⁺.

### Step c): 6-{4-[(2-{[tert-Butyl(dimethyl)silyl]oxy}ethyl)amino]phenyl}-1-(4-methoxyphenyl)-7-oxo-4,5,6,7-tetrahydro-1H-pyrazolo[3,4-c]pyridine-3-carboxamide

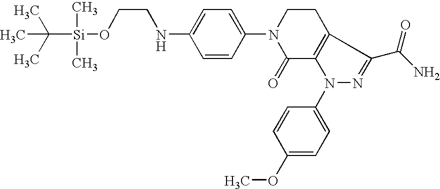

Under argon and at RT, 106 mg (0.28 mmol, 1.1 eq.) of O-(7-azabenzotriazol-1-yl)-N,N,N′,N′-tetramethyluronium hexafluorophosphate (HATU), 39 mg (0.51 mmol, 2 eq.) of ammonium acetate and 97 μl (0.56 mmol, 2.2 eq.) of N,N-diisopropylethylamine are added to a solution of 195 mg (70% pure, 0.25 mmol) of 6-{4-[(2-{[tert-butyl(dimethyl)silyl]oxy}ethyl)amino]phenyl}-1-(4-methoxyphenyl)-7-oxo-4,5,6,7-tetrahydro-1H-pyrazolo[3,4-c]pyridine-3-carboxylic acid in 10 ml of dimethylformamide. The reaction mixture is stirred at RT for 21 h and concentrated under reduced pressure. The residue is taken up in a mixture of saturated aqueous ammonium chloride solution and ethyl acetate. After phase separation, the aqueous phase is extracted twice with ethyl acetate. The combined organic phases are washed with water and with saturated aqueous sodium chloride solution, dried over sodium sulphate, filtered and concentrated under reduced pressure. The title compound is isolated by preparative RP-HPLC (CromSil C18, acetonitrile/water gradient).

Yield: 52 mg (38% of theory)

LC-MS (method 3): Rₜ=2.90 min;

MS (ESIpos): m/z=536 [M+H]⁺;

¹H-NMR (400 MHz, DMSO-d₆): δ=7.68 (s, 1H), 7.47 (d, 2H), 7.39 (s, 1H), 7.02 (d, 2H), 6.98 (d, 2H), 6.55 (d, 2H), 5.54 (t, 1H), 3.91 (t, 2H), 3.79 (s, 3H), 3.69 (t, 2H), 3.19-3.08 (m, 4H), 0.85 (s, 9H), 0.0 (s, 6H).

### Step d): 6-{4-[(2-{[tert-Butyl(dimethyl)silyl]oxy}ethyl)(cyano)amino]phenyl}-1-(4-methoxyphenyl)-7-oxo-4,5,6,7-tetrahydro-1H-pyrazolo[3,4-c]pyridine-3-carboxamide

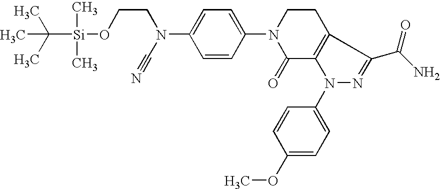

Under argon and at RT, 23 mg (0.28 mmol, 3 eq.) of sodium bicarbonate and 46 μl of a cyanogen bromide solution (3 M in dichloromethane, 0.14 mmol, 1.5 eq.) are added to a solution of 51 mg (0.09 mmol) of 6-{4-[(2-{[tert-butyl(dimethyl)silyl]oxy}ethyl)amino]phenyl}-1-(4-methoxyphenyl)-7-oxo-4,5,6,7-tetrahydro-1H-pyrazolo[3,4-c]pyridine-3-carboxamide in 2.9 ml of tetrahydrofuran. The reaction mixture is stirred at 40° C. for 4 d. After addition of water/dichloromethane and phase separation, the aqueous phase is extracted twice with dichloromethane. The combined organic phases are washed with saturated aqueous sodium bicarbonate solution and with saturated aqueous sodium chloride solution, dried over sodium sulphate, filtered and concentrated under reduced pressure. The title compound is isolated by preparative RP-HPLC (CromSil C18, acetonitrile/water gradient).

Yield: 37 mg (72% of theory)

LC-MS (method 3): Rₜ=2.75 min;

MS (ESIpos): m/z=561 [M+H]⁺;

¹H-NMR (400 MHz, DMSO-d₆): δ=7.73 (s, 1H), 7.51 (d, 2H), 7.45 (s, 1H), 7.40 (d, 2H), 7.21 (d, 2H), 7.01 (d, 2H), 4.03 (t, 2H), 3.85 (s, 4H), 3.81 (s, 3H), 3.21 (t, 2H), 0.86 (s, 9H), 0.0 (s, 6H).

### Step e): 6-[4-(2-Imino-1,3-oxazolidin-3-yl)phenyl]-1-(4-methoxyphenyl)-7-oxo-4,5,6,7-tetrahydro-1H-pyrazolo[3,4-c]pyridine-3-carboxamide methanesulphonate

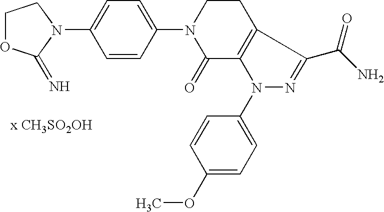

At RT, 8 μl (0.12 mmol, 2.1 eq.) of methanesulphonic acid are added to a solution of 34 mg (0.06 mmol) of 6-{4-[(2-{[tert-butyl(dimethyl)silyl]oxy}ethyl)(cyano)amino]phenyl}-1-(4-methoxyphenyl)-7-oxo-4,5,6,7-tetrahydro-1H-pyrazolo[3,4-c]pyridine-3-carboxamide in 2 ml of acetonitrile. The reaction mixture is stirred at RT for 20 h, and 2 ml of diethyl ether are added. The precipitate formed is filtered off, washed with diethyl ether and dried under reduced pressure.

Yield: 14.4 mg (46% of theory)

HPLC (method 5): Rₜ=3.43 min;

MS (ESIpos): m/z=447 [M+H]⁺ (free base);

¹H-NMR (400 MHz, DMSO-d₆): δ=9.60 (br. s, 1H), 8.90 (br. s, 1H), 7.74 (s, 1H), 7.59-7.51 (m, 4H), 7.50 (d, 2H), 7.44 (s, 1H), 7.00 (d, 2H), 4.84 (t, 2H), 4.25 (t, 2H), 4.08 (t, 2H), 3.80 (s, 3H), 3.23 (t, 2H), 2.28 (s, 3H)

### B. EVALUATION OF THE PHARMACOLOGICAL ACTIVITY

The compounds according to the invention act in particular as selective inhibitors of blood coagulation factor Xa and do not, or only at significantly higher concentrations, inhibit other serine proteases, such as plasmin or trypsin.

“Selective” are those inhibitors of the blood coagulation factor Xa in which the IC₅₀ values for the factor Xa inhibition are lower by a factor of at least 100 compared to the IC₅₀ values for the inhibition of other serine proteases, in particular plasmin and trypsin, where, with respect to the test methods for the selectivity, reference is made to the test methods, described below, of Examples B.a.1) and B.a.2).

The advantageous pharmacological properties of the compounds according to the invention can be determined by the following methods:

### a) Test Descriptions (In Vitro)

### a.1) Determination of the Factor Xa Inhibition:

The enzymatic activity of human factor Xa (FXa) is measured using the conversion of a chromogenic substrate specific for FXa. Factor Xa cleaves p-nitroaniline from the chromogenic substrate. The determinations are carried out in microtitre plates as follows:

The test substances, in various concentrations, are dissolved in DMSO and incubated for 10 minutes at 25° C. with human FXa (0.5 mmol/l dissolved in 50 mmol of Tris buffer [C,C,C-tris(hydroxymethyl)aminomethane], 150 mmol/l of NaCl, 0.1% BSA [bovine serum albumin], pH=8.3). Pure DMSO is used as control. The chromogenic substrate (150 μmol/l of Pefachrome® FXa from Pentapharm) is then added. After an incubation time of 20 minutes at 25° C., the extinction at 405 nm is determined. The extinctions of the test mixtures containing the test substance are compared with the control mixtures without test substance, and the IC₅₀ values are calculated from these data.

Representative activity data from this test are listed in Table 1 below:

TABLE 1

| Example No. | IC₅₀ [nM] |
|---|---|
| 1 | 0.8 |

### a.2) Determination of the Selectivity:

To demonstrate the selective FXa inhibition, the test substances are examined for their inhibition of other human serine proteases, such as trypsin and plasmin. To determine the enzymatic activity of trypsin (500 mU/ml) and plasmin (3.2 nmol/l), these enzymes are dissolved in Tris buffer (100 mmol/l, 20 mmol/l of CaCl₂, pH=8.0) and incubated with test substance or solvent for 10 minutes. The enzymatic reaction is then started by addition of the appropriate specific chromogenic substrates (Chromozym Trypsin® and Chromozym Plasmin®; from Roche Diagnostics), and after 20 minutes the extinction is determined at 405 nm. All determinations are carried out at 37° C. The extinctions of the test batches with test substance are compared to the control samples without test substance, and the IC₅₀ values are calculated from these data.

### a.3) Determination of the Anticoagulatory Activity:

The anticoagulatory activity of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

### b) Determination of the Antithrombotic Activity (In Vivo)

### b.1) Arteriovenous Shunt Model (Rabbit):

Fasting rabbits (strain: Esd: NZW) are anaesthetized by intramuscular administration of Rompun/Ketavet solution (5 mg/kg and 40 mg/kg, respectively). Thrombus formation is initiated in an arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

### c) Solubility Assay

Reagents required:
PBS buffer pH 7.4: 90.00 g of NaCl p.a. (for example Merck Art. No. 1.06404.1000), 13.61 g of KH₂PO₄ p.a. (for example Merck Art. No. 1.04873.1000) and 83.35 g of 1N NaOH (for example Bernd Kraft GmbH Art. No. 01030.4000) are weighed into a 1 l measuring flask, the flask is filled with water and the mixture is stirred for about 1 hour.
Acetate buffer pH 4.6: 5.4 g of sodium acetate×3H₂O p.a. (for example Merck Art. No. 1.06267.0500) are weighed into a 100 ml measuring flask and dissolved in 50 ml of water, 2.4 g of glacial acetic acid are added, the mixture is made up to 100 ml with water, the pH is checked and, if required, adjusted to pH 4.6.
Dimethyl sulphoxide (for example Baker Art. No. 7157.2500)
Distilled water

### Preparation of the Calibration Solutions:

Preparation of the stock solution of calibration solutions: About 0.5 mg of the active compound are weighed accurately into a 2 ml Eppendorf Safe-Lock tube (Eppendorf Art. No. 0030 120.094), DMSO is added to a concentration of 600 μg/ml (for example 0.5 mg of active compound+833 μl of DMSO) and the mixture is vortexed until everything has gone into solution.

Calibration solution 1 (20 μg/ml): 1000 μl of DMSO are added to 34.4 μl of the stock solution, and the mixture is homogenized.

Calibration solution 2 (2.5 μg/ml): 700 μl of DMSO are added to 100 μl of calibration solution 1, and the mixture is homogenized.

### Preparation of the Sample Solutions:

Sample solution for solubilities of up to 10 g/l in PBS buffer pH 7.4: About 5 mg of the active compound are weighed accurately into a 2 ml Eppendorf Safe-Lock tube (Eppendorf Art. No. 0030 120.094), and PBS buffer pH 7.4 is added to a concentration of 5 g/l (for example 5 mg of active compound+500 μl of PBS buffer pH 7.4).

Sample solution for solubilities of up to 10 g/l in acetate buffer pH 4.6: About 5 mg of the active compound are weighed accurately into a 2 ml Eppendorf Safe-Lock tube (Eppendorf Art. No. 0030 120.094), and acetate buffer pH 4.6 is added to a concentration of 5 g/l (for example 5 mg of active compound+500 μl of acetate buffer pH 4.6).

Sample solution for solubilities of up to 10 g/l in water: About 5 mg of the active compound are weighed accurately into a 2 ml Eppendorf Safe-Lock tube (Eppendorf Art. No. 0030 120.094), and water is added to a concentration of 5 g/l (for example 5 mg of active compound+500 μl of water).

### Practice:

The sample solutions prepared in this manner are shaken at 1400 rpm in a temperature-adjustable shaker (for example Eppendorf Thermomixer comfort Art. No. 5355 000.011 with interchangeable block Art. No. 5362.000.019) at 20° C. for 24 hours. In each case 180 μl are taken from these solutions and transferred into Beckman Polyallomer centrifuge tubes (Art. No. 343621). These solutions are centrifuged at about 223 000*g for 1 hour (for example Beckman Optima L-90K ultracentrifuge with type 42.2 Ti rotor at 42 000 rpm). From each of the sample solutions, 100 μl of the supernatant are removed and diluted 1:5, 1:100 and 1:1000 with the respective solvent used (water, PBS buffer 7.4 or acetate buffer pH 4.6). From each dilution, a sample is transferred into a vessel suitable for HPLC analysis.

### Analysis:

The samples are analyzed by RP-HPLC. Quantification is carried out using a two-point calibration curve of the test compound in DMSO. The solubility is expressed in mg/l.

### Analysis Sequence:

1. Calibration solution 2.5 mg/ml
2. Calibration solution 20 μg/ml
3. Sample solution 1:5
4. Sample solution 1:100
5. Sample solution 1:1000
   HPLC method for Acids:

Agilent 1100 with DAD (G1315A), quat. pump (G1311A), autosampler CTC HTS PAL, degasser (G1322A) and column thermostat (G1316A); column: Phenomenex Gemini C18, 50×2 mm, 5μ; temperature: 40° C.; mobile phase A: water/phosphoric acid pH 2; mobile phase B: acetonitrile; flow rate: 0.7 mL/min; gradient: 0-0.5 min 85% A, 15% B; ramp: 0.5-3 min 10% A, 90% B; 3-3.5 min 10% A, 90% B; ramp: 3.5-4 min 85% A, 15% B; 4-5 min 85% A, 15% B.

### HPLC Method for Bases:

Agilent 1100 with DAD (G1315A), quat. pump (G1311A), autosampler CTC HTS PAL, degasser (G1322A) and column thermostat (G1316A); column: VDSoptilab Kromasil 100 C18, 60×2.1 mm, 3.5μ; temperature: 30° C.; mobile phase A: water+5 ml perchloric acid/l; mobile phase B: acetonitrile; flow rate: 0.75 ml/min; gradient: 0-0.5 min 98% A, 2% B; ramp: 0.5-4.5 min 10% A, 90% B; 4.5-6 min 10% A, 90% B; ramp: 6.5-6.7 min 98% A, 2% B; 6.7-7.5 min 98% A, 2% B.

### C. EXEMPLARY EMBODIMENTS OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Preparation:

The mixture of the compound according to the invention, lactose and starch is granulated with a 5% strength solution (m/m) of PVP in water. The granules are dried and then mixed with the magnesium stearate for 5 minutes. This mixture is compressed using a conventional tablet press (see above for format of the tablet). As guideline, a compressive force of 15 kN is used for the compression.

### Oral Suspension:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension are equivalent to a single dose of 100 mg of the compound according to the invention.

### Preparation:

The Rhodigel is suspended in ethanol, and the compound according to the invention is added to the suspension. The water is added while stirring. The mixture is stirred for about 6 h until the swelling of the Rhodigel is complete.

### Oral Solution:

### Composition:

500 mg of the compound according to the invention, 2.5 g of polysorbate and 97 g of polyethylene glycol 400.20 g of oral solution are equivalent to a single dose of 100 mg of the compound according to the invention.

### Production:

The compound according to the invention is suspended in the mixture of polyethylene glycol and polysorbate while stirring. Stirring is continued until the compound according to the invention is completely dissolved.

i.v. Solution:

The compound according to the invention is dissolved at a concentration below saturation solubility in a physiologically acceptable solvent (for example isotonic sodium chloride solution, glucose solution 5% and/or PEG 400 solution 30%). The solution is sterilized by filtration and filled into sterile and pyrogen-free injection containers.

1. Compound of the formula 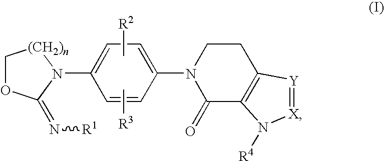
in which
n represents the number 1, 2 or 3,
X represents N or CH,
Y represents N or CR⁵,
where
R⁵ represents hydrogen, cyano, trifluoromethyl, trifluoromethoxy, hydroxy, hydroxymethyl, hydroxyethyl, amino, aminomethyl, aminoethyl, C₁-C₄-alkyl, C₁-C₄-alkoxy, C₁-C₄-alkoxymethyl, C₁-C₄-alkoxyethyl, C₁-C₄-alkylamino, C₁-C₄-alkylaminomethyl, C₁-C₄-alkylaminoethyl, C₃-C₆-cycloalkyl, hydroxycarbonyl, hydroxycarbonylmethyl, hydroxycarbonylethyl, aminocarbonyl, amino carbonylmethyl, amino carbonylethyl, C₁-C₄-alkoxycarbonyl, C₁-C₄-alkoxycarbonylmethyl, C₁-C₄-alkoxycarbonylethyl, C₁-C₄-alkylcarbonyloxy, C₁-C₄-alkylaminocarbonyl, C₁-C₄-alkylaminocarbonylmethyl, C₁-C₄-alkylaminocarbonylethyl, C₁-C₄-alkylcarbonylamino, C₁-C₄-alkylcarbonylaminomethyl, C₁-C₄-alkylcarbonylaminoethyl, C₁-C₄-alkylcarbonyl(C₁-C₄-alkyl)amino, C₁-C₄-alkylcarbonyl(C₁-C₄-alkyl)aminomethyl, C₁-C₄-alkylcarbonyl(C₁-C₄-alkyl)aminoethyl, aminosulphonyl, C₁-C₄-alkylaminosulphonyl, C₁-C₄-alkylsulphonyl or tetrazolyl,
R¹ represents hydrogen, cyano, hydroxy, C₁-C₄-alkyl, C₁-C₄-alkylcarbonyl, C₃-C₆-cycloalkylcarbonyl, phenylcarbonyl, 4 to 7-membered heterocyclyl carbonyl or 5 to 6-membered heteroarylcarbonyl,
R² represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R³ represents hydrogen, fluorine, chlorine, cyano, hydroxy, amino, trifluoromethyl, trifluoromethoxy, C₁-C₄-alkyl, C₁-C₄-alkoxy, C₁-C₄-alkoxymethyl, C₁-C₄-alkylamino, C₃-C₆-cycloalkyl, aminocarbonyl, C₁-C₄-alkoxycarbonyl or C₁-C₄-alkylaminocarbonyl,
R⁴ represents phenyl, pyridyl, pyrazinyl, pyrimidinyl or pyridazinyl,
where phenyl is substituted by a substituent R⁶ and a substituent R⁷,
where
R⁶ represents hydrogen, fluorine, chlorine, cyano, amino, ethynyl, aminomethyl, C₁-C₄-alkyl, C₁-C₄-alkoxy, C₃-C₆-cycloalkyl, C₃-C₆-cycloalkyloxy, aminocarbonyl, C₁-C₄-alkylaminocarbonyl, aminosulphonyl or methylcarbonylaminosulphonyl,
and
R⁷ represents hydrogen, fluorine, chlorine, cyano, trifluoromethyl, trifluoromethoxy, C₁-C₄-alkyl, C₁-C₄-alkoxy, C₃-C₆-cycloalkyl or C₃-C₆-cycloalkyloxy,
and
where pyridyl, pyrazinyl, pyrimidinyl and pyridazinyl are substituted by a substituent R⁸ and/or a substituent R⁹ or by two different substituents R⁸ or by two different substituents R⁹,
where
R⁸ is attached to a carbon atom which is not adjacent to a nitrogen atom in the ring and represents hydrogen, fluorine, chlorine, cyano, amino, ethynyl, aminomethyl, C₁-C₄-alkyl, C₁-C₄-alkoxy, C₃-C₆-cycloalkyl or C₃-C₆-cycloalkyloxy,
and
R⁹ is attached to a carbon atom which is adjacent to a nitrogen atom in the ring and represents hydrogen, amino, ethynyl, C₁-C₄-alkyl, C₁-C₄-alkylamino, C₃-C₆-cycloalkyl or C₃-C₆-cycloalkylamino,
or one of its salts, its solvates or the solvates of its salts.

2. Compound according to claim 1, characterized in that
n represents the number 1, 2 or 3,
X represents N or CH,
Y represents N or CR⁵,
where
R⁵ represents hydrogen, cyano, trifluoromethyl, hydroxymethyl, aminomethyl, C₁-C₄-alkyl, C₁-C₄-alkoxy, C₁-C₄-alkoxymethyl, C₁-C₄-alkylamino, C₁-C₄-alkylaminomethyl, C₃-C₆-cycloalkyl, hydroxycarbonyl, hydroxycarbonylmethyl, aminocarbonyl, aminocarbonylmethyl, C₁-C₄-alkoxycarbonyl, C₁-C₄-alkoxycarbonylmethyl, C₁-C₄-alkylaminocarbonyl, C₁-C₄-alkylaminocarbonylmethyl, C₁-C₄-alkylcarbonylamino, C₁-C₄-alkylcarbonylaminomethyl, C₁-C₄-alkylcarbonyl-(C₁-C₄-alkyl)amino, C₁-C₄-alkylcarbonyl(C₁-C₄-alkyl)aminomethyl, aminosulphonyl, C₁-C₄-alkylaminosulphonyl, C₁-C₄-alkylsulphonyl or tetrazolyl,
R¹ represents hydrogen, cyano, hydroxy, or C₁-C₄-alkyl,
R² represents hydrogen, fluorine, chlorine, cyano, hydroxy, C₁-C₄-alkyl or C₁-C₄-alkoxy,
R³ represents hydrogen, fluorine, chlorine, cyano, hydroxy, C₁-C₄-alkyl, C₁-C₄-alkoxy, C₁-C₄-alkoxymethyl, cyclopropyl, aminocarbonyl, C₁-C₄-alkoxycarbonyl or C₁-C₄-alkylaminocarbonyl,
R⁴ represents phenyl, pyridyl, pyrazinyl, pyrimidinyl or pyridazinyl,
where phenyl is substituted by a substituent R⁶ and a substituent R⁷,
where
R⁶ represents hydrogen, fluorine, chlorine, cyano, ethynyl, aminomethyl, methyl, ethyl, methoxy, ethoxy, aminocarbonyl, aminosulphonyl or methylcarbonylaminosulphonyl,
R⁷ represents hydrogen, fluorine, chlorine, cyano, trifluoromethyl, trifluoromethoxy, methyl, ethyl, methoxy or ethoxy,
and
where pyridyl, pyrazinyl, pyrimidinyl and pyridazinyl are substituted by a substituent R⁸ and/or a substituent R⁹ or by two different substituents R⁸ or by two different substituents R⁹,
where
R⁸ is attached to a carbon atom which is not adjacent to a nitrogen atom in the ring and represents hydrogen, fluorine, chlorine, cyano, ethynyl, aminomethyl, methyl, ethyl, methoxy or ethoxy,
R⁹ is attached to a carbon atom which is adjacent to a nitrogen atom in the ring, and represents hydrogen, amino, ethynyl, methyl, ethyl, methylamino or dimethylamino,
and
where R⁸, if present, is attached to the para-position and R⁹, if present, is attached to the meta-position to the point of attachment of the phenyl ring, or
where R⁸, if present, is attached to the meta-position and R⁹, if present, is attached to the para-position to the point of attachment of the phenyl ring.

3. Compound according to claim 1, wherein
n represents the number 1 or 2,
X represents N,
Y represents CR⁵,
where
R⁵ represents hydrogen, cyano, trifluoromethyl, hydroxymethyl, aminomethyl, methoxy, methoxymethyl, methylaminomethyl, dimethylaminomethyl, hydroxycarbonyl, aminocarbonyl, methoxycarbonyl, ethoxycarbonyl, methylaminocarbonyl, dimethylaminocarbonyl or tetrazolyl,
R¹ represents hydrogen,
R² represents hydrogen,
R³ represents hydrogen, fluorine, chlorine, cyano, methyl, ethyl, n-propyl, methoxy, ethoxy or methoxymethyl,
R⁴ represents 4-methoxyphenyl, 3-chlorophenyl, 3-chloro-4-fluorophenyl, 4-chlorophenyl, 3-methylphenyl, 3-methyl-4-fluorophenyl, 4-methylphenyl, 4-ethylphenyl, 3-aminomethylphenyl, 3-aminomethyl-4-fluorophenyl, 3-aminocarbonylphenyl, 5-chloropyridin-2-yl, 5-methylpyridin-2-yl, 5-ethylpyridin-2-yl, 5-methoxypyridin-2-yl, 5-chloropyrimidin-2-yl, 5-methylpyrimidin-2-yl, 5-ethylpyrimidin-2-yl, 5-methoxypyrimidin-2-yl, 6-methylpyridin-3-yl, 6-ethylpyridin-3-yl, 6-methylpyridazin-3-yl or 6-ethylpyridazin-3-yl.

4. Compound according to claim 1 wherein
n represents the number 1,
x represents N,
Y represents CR⁵,
where
R⁵ represents aminocarbonyl, methoxycarbonyl or ethoxycarbonyl, represents hydrogen,
R¹ represents hydrogen,
R² represents hydrogen,
R³ represents hydrogen, fluorine, chlorine, cyano, methyl or methoxy,
R⁴ represents 4-methoxyphenyl.

5. Process for preparing a compound of the formula (I) or one of its salts, its solvates or the solvates of its salts according to claim 1, wherein
[A] a compound of the formula 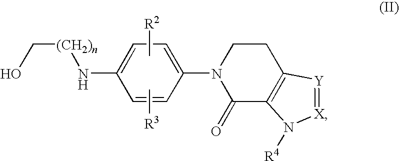
in which n, X, Y, R², R³ and R⁴ have the meaning given in claim 1
is reacted in an inert solvent in the presence of an acid with cyanogen bromide to give a compound of the formula (I) in which R¹ represents hydrogen,
or
[B] a compound of the formula 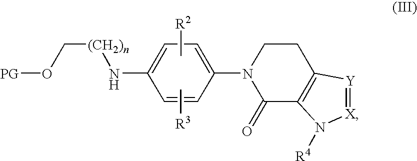
in which n, X, Y, R², R³ and R⁴ have the meaning given in claim 1, and
PG represents a hydroxyl protective group, preferably trimethylsilyl or tert-butyldimethylsilyl,
is reacted in a three-step process initially in an inert solvent with cyanogen bromide, preferably in the presence of a base, to give a compound of the formula 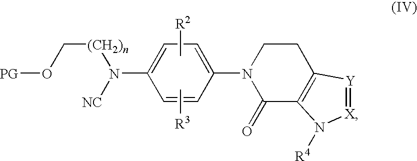
in which n, X, Y, R², R³ and R⁴ have the meaning given in claim 1, and
PG represents a hydroxyl protective group, preferably trimethylsilyl or tert-butyldimethylsilyl,
and then by removal of the protective group PG to give a compound of the formula 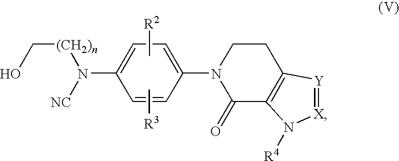
in which n, X, Y, R², R³ and R⁴ have the meaning given in claim 1,
and in the third step the compound of the formula (V) is cyclized in an inert solvent in the presence of an acid to give a compound of the formula (I) in which R¹ represents hydrogen,
or
[C] the compound of the formula (II) is reacted in the first step with a compound of the formula 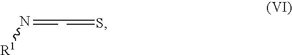
in which
R¹ represents C₁-C₄-alkyl, C₁-C₄-alkylcarbonyl, C₃-C₆-cycloalkylcarbonyl, phenylcarbonyl, 4- to 7-membered heterocyclylcarbonyl or 5- or 6-membered heteroarylcarbonyl,
and cyclized in the second step,
or
[D] a compound of the formula (II) is reacted with a compound of the formula 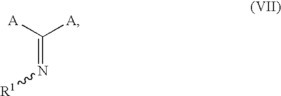
in which
R¹ represents cyano or C₁-C₄-alkyl, and
A represents a leaving group, preferably phenoxy or methylthio,
or
[E] the compound of the formula (I) in which R¹ represents hydrogen is reacted with hydroxylamine hydrochloride to give a compound of the formula (I) in which R¹ represents hydroxyl.

6. Compound according to claim 1 for the treatment and/or prophylaxis of diseases.

7. (canceled)

8. (canceled)

9. (canceled)

10. A pharmaceutical composition comprising a compound according to claim 1 in combination with an inert non-toxic pharmaceutically acceptable auxiliary.

11. The pharmaceutical composition of claim 10 further comprising another active compound.

12. (canceled)

13. A method for the treatment and/or prophylaxis of thromboembolic disorders in humans and animals comprising administering an anticoagulatory effective amount of at least one compound according to claim 1.

14. A method for preventing blood coagulation in vitro, characterized in that an anticoagulatory effective amount of a compound according to claim 1 is added.
